(19) United States
(12) Patent Application Publication
(10) Pub. No.: US 2005/0118204 A1
(43) Pub. Date: Jun. 2, 2005

(54) Methods for controlling membrane permeability of a membrane permeable substance and screening methods for a membrane permeable substance

(76) Inventors: Kazutami Sakamoto, Tokyo (JP); Yoshinobu Takino, Kawasaki-shi (JP); Kazuko Ogasahara, Tokyo (JP)

Correspondence Address: NATH & ASSOCIATES, PLLC, Sixth Floor, 1030 15th Street, N.W., Washington, DC, 20005, US

(21) Appl. No.: 10/948,755

(22) Filed: Sep. 24, 2004

Related U.S. Application Data
(60) Provisional application No. 60/517,980, filed on Nov. 5, 2003.
(60) Provisional application No. 60/517,889, filed on Nov. 7, 2003.

(30) Foreign Application Priority Data
Nov. 4, 2003 (JP) 2003-374224
Sep. 25, 2003 (JP) 2003-332894

(51) Int. Cl. A61K 38/00; A61K 9/00; C12Q 1/00

(52) U.S. Cl. 424/400; 435/4; 514/2

(57) ABSTRACT

The present invention relates to a method for controlling membrane permeability of a membrane permeable substance, and more particularly it relates to a method for controlling membrane permeability of a membrane permeable substance by changing the curvature of the membrane. The invention also relates to a screening method for a membrane permeable substance, and more particularly, it relates to a screening method for a membrane permeable substance, in which the change of curvature of the membrane and/or the phase change of the membrane, which occurs upon adding a test substance, is detected to evaluate the membrane permeability of the test substance.

### BACKGROUND OF THE INVENTION

1. Field of the Invention

The present invention relates to a method for controlling membrane permeability of a membrane permeable substance, and more particularly it relates to a method for controlling membrane permeability of a membrane permeable substance by changing the curvature of the membrane.

The invention also relates to a screening method for a membrane permeable substance, and more particularly, it relates to a screening method for a membrane permeable substance, in which the change of curvature of the membrane and/or the phase change of the membrane, which occurs upon adding a test substance, is detected to evaluate the membrane permeability of the test substance.

2. Description of the Related Art

A membrane, such as a biological membrane, functions as a separation of an interior surrounded by the membrane from an exterior thereof to execute normal biological functions of the interior. The membrane does not completely isolate the interior from the exterior, but for example, a cell membrane has mechanisms for executing exchange of substances, information and energy to the exterior of the cell, and for executing various metabolic reactions. Specifically, a cell membrane has particular transporters and receptors, and executes selective incorporation of necessary substances and discharge of metabolic products owing to the functions of the transporters and receptors.

In recent years, however, water soluble membrane permeable substances have become known which penetrate a membrane, such as a cell membrane, without any particular transporter or receptor. Examples of such a membrane permeable substance include a peptide having a plurality of guanidino side chains, amidino side chains or amino groups, and applications thereof to drug delivery have been proposed, in which the membrane permeable substance is combined with a drug or the like and penetrates through a biological membrane along with the drug (as disclosed in U.S. Pat. No. 6,495,663 (JP-T-2002-502376), U.S. Patent Publication No. 2004/0074504A (JP-T-2003-501393) and U.S. Patent Publication No. 2003/0022831A (JP-T-2003-507438), wherein the term “JP-T” as used herein means a published Japanese translation of a PCT patent application).

However, only a small number of kinds of membrane permeable substances are known, and the application ranges thereof are restricted. Furthermore, in order to control the permeability of such substances, it is necessary to change the concentration and the application time of the membrane permeable substances, and moreover to change the structures of the membrane permeable substances themselves. In many cases, the concentration and the application time cannot be freely changed due to properties of a drug required to penetrate the membrane, and it is not easy to change the structures of the membrane permeable substances to correspond to the properties of the drug to be applied. Accordingly, there is a need to develop a convenient method for controlling permeability in order to use such substances in a drug delivery system, etc.

It is also useful to investigate for novel membrane permeable substances controlling the permeability of a drug. However, no useful method has been known for screening for such membrane permeable substances, and it is necessary that the substances actually penetrate through membranes to confirm the permeability thereof. Specifically, it is necessary, for example, that the concentration of the permeable substance is measured on the side where the substance is received by using HPLC (high-performance liquid chromatography), a part of the test substance is replaced with a radioactive isotope to detect radiation therefrom, and a fluorescent substance is bonded to the test substance to measure the fluorescence intensity. However, these methods include complicated operations, and in some cases, these methods cannot be applied depending on the properties of the test sample.

An object of the invention is to provide such a method as is needed, which can conveniently screen a membrane permeable substance.

### SUMMARY OF THE INVENTION

As a result of analysis of the membrane permeation mechanism of the membrane permeable substance made by the inventors, it has been found that the membrane permeation occurs according to the following mechanism.

In general, a higher order structure, such as micelle, hexagonal, cubic and lamellar, formed through self-organization of an amphiphilic substance (surface active agent) is specified by a stable structure in an equilibrium system depending on the structure, the composition and the concentration of the amphiphilic substance, the species and the amount of the additional substance, the temperature, and so forth. The inventive membranes include a biological membrane in a lamellar phase constituted by an amphiphilic substance which maintains a stable structure in an equilibrium system.

The established theory is that the difference between a lamellar liquid crystal phase and a cubic liquid crystal phase (V₁ phase) in an equilibrium system is generally determined by the geometric proportion of the hydrophilic part and the hydrophobic part of the amphiphilic substance constituting the membrane expressed as a packing parameter, i.e., determined by the curvature of the membrane, which has been verified in various systems. It has been known that in the case where a lamellar liquid crystal layer having zero membrane curvature is applied under conditions providing a positive curvature (convexity toward an aqueous solution phase outside the membrane) by addition of a substance or a physical change of a circumstance, phase transition to a cubic liquid crystal phase (V₁ phase) with the hydrophilic groups being outside occurs, and when the layer is applied with a condition providing a negative curvature (concavity toward an aqueous solution phase outside the membrane), phase transition to a cubic liquid crystal phase (V₂ phase) with the hydrophobic groups being outside occurs, as shown in FIG. 1. The phase transition from the lamellar liquid crystal phase to the cubic liquid crystal phase occurs with a slight curvature change, and energy required for the phase transition is significantly small (as described in H. Kunieda and K. Aramaki, Oleoscience, vol. 1, p. 179 (2001), and H. Kunieda and K. Sakamoto, Kagaku Binran (Chemistry Handbook) 6th ed., Applied Chemistry Volume II, Chapter 19.4 Surface Active Agent, P 1019, edited by The Chemical Society of Japan, published by Maruzen Co., Ltd. (2003)).

The cubic phase (V₁ phase and V₂ phase) formed through transition from the lamellar phase has a bicontinuous form, i.e., the aqueous phase (hydrophilic group phase) or the oily phase (hydrophobic group phase) is three-dimensionally continuous over the entire system to provide such a structure that the two phases are separated by an amphiphilic lipid membrane. The cubic phase of this type has a three-dimensional periodic structure minimizing a crystallographically classified surface area, and the presence thereof has been confirmed in various membrane tissues in living organisms (as described in K. Larsson, J. Phys. Chem., vol. 93, p. 7304-7314 (1989).

As a model for resolving the curvature change partially occurring in a bimolecular membrane, which is a lamellar phase separating an interior and an exterior, like a biological membrane, a model referred to as a mesh type has been proposed that has pores for minimizing the surface area in the cubic phase, and the presence thereof has been confirmed in a model lipid system (as shown in FIG. 2), described in S. T. Hyde and G. E. Schroder, Current Opinion in Colloid Interface Science, vol. 8, p. 5-14 (2003). However, formation of pores in an equilibrium system impairs the separating function of the membrane, and a model avoiding the problem has been proposed by Larsson (as shown in FIG. 3). In this case, pores locally formed in a cubic phase are clogged with large membrane protein on the hydrophilic side to stabilize the system (as described in K. Larsson, J. Phys. Chem., vol. 93, p. 7304-7314 (1989). However, this model is an important mechanism relating to controlling substance permeation by the protein having been present in the membrane, so this is not applicable to the permeation mechanism of the inventive membrane permeable substance dissolved in the external aqueous solution.

The inventors have demonstrated that the permeation phenomenon of an inventive water soluble membrane permeable substance can be described as local phase transition as a dynamic phenomenon. Specifically, it has been well known that even in a lamellar liquid crystal with zero curvature as an equilibrium system, fluctuation due to molecular motion within minute period of time locally causes such a repeated slight curvature change within a range causing no phase transition. In the case where the membrane is in contact with a water soluble membrane permeable substance which positively change the membrane curvature to be convex, a change corresponding to phase transition to the cubic liquid crystal phase (V₁ phase) locally occurs as a dynamic fluctuation phenomenon at the contact part. In the case where the additional amount of the water soluble membrane permeable substance is within a certain amount, no phase transition occurs over the system as an equilibrium system as a whole. As a result, permeation of the substance is enabled from the side donating the membrane permeable substance (outside of the membrane) to the side accepting the membrane permeable substance (inside the membrane) at the part where the cubic liquid crystal phase formed locally, while the structure of the lipid bimolecular membrane is maintained over the membrane. The tendency in change of the curvature of the membrane is not uniform but depends on the species of the membrane, the species of the permeable substance and the mutual action between them.

Therefore, the membrane permeability of the membrane permeable substance can be further controlled by enhancing or inhibiting the function of changing the membrane curvature by the membrane permeable substance. Based on these findings, the inventors have found that the membrane permeability of the membrane permeable substance can be controlled by changing the membrane curvature by different methods, and thus the invention has been completed.

The inventors also have found that the membrane permeability of the substance can be evaluated by detecting the change in the membrane curvature, and the membrane permeability of the substance can be evaluated by detecting the phase change of the membrane, and thus the invention has been completed.

Accordingly, the invention relates to a method for controlling permeability of a membrane by a membrane permeable substance, comprising the step of changing a membrane curvature.

The invention also relates to a method for enhancing permeability of a membrane, comprising the step of changing a membrane curvature, in a direction making the membrane convex toward a side donating a membrane permeable substance.

The invention also relates to a method for suppressing permeability of a membrane, comprising the step of changing a membrane curvature, in a direction making the membrane concave toward a side of said membrane donating a membrane permeable substance.

The aforementioned methods optionally may contain the step of changing the membrane curvature by changing at least one factor selected from the group consisting of osmotic pressure, temperature, electromagnetic field and pH.

The aforementioned methods optionally may comprise the step of adding a substance which is capable of changing the membrane curvature.

The invention further relates to a method for screening a membrane permeable substance, comprising the steps of:
adding a test substance to a solvent dispersing cell bodies enclosed with a membrane; and
detecting a change of the membrane curvature of the cell bodies enclosed by a membrane before and after the addition of the test substance.

The invention still further relates to a method for screening a membrane permeable substance, comprising the steps of:
retaining a solvent in a first chamber and a second chamber separated from each other with a membrane;
adding a test substance to the first chamber; and
detecting a change of the membrane curvature before and after the addition of the test substance.

The invention further relates to a method for screening a membrane permeable substance, comprising the steps of:
adding a test substance to a solvent dispersing cell bodies enclosed with a membrane; and
detecting a phase change of the membrane of the cell bodies enclosed by a membrane before and after the addition of the test substance.

According to the invention, the permeability of the membrane permeable substance through the membrane can be conveniently adjusted, and the membrane permeable substance can be conveniently screened.

### BRIEF DESCRIPTION OF THE DRAWINGS

FIG. 1 is a diagram showing a phase change from a lamellar liquid crystal phase to a cubic liquid crystal phase.

FIG. 2 is a diagram showing a model proposed by Hyde, et al.

FIG. 3 is a diagram showing a model proposed by Larsson, et al.

FIG. 4A is a graph showing the relationship between an osmotic pressure and the curvature of an erythrocyte, in a 70% hypotonic phosphate buffer solution.

FIG. 4B is a graph showing the relationship between an osmotic pressure and the curvature of an erythrocyte, in an isotonic phosphate buffer solution.

FIG. 4C is a graph showing the relationship between an osmotic pressure and the curvature of an erythrocyte, in a 130% hypertonic phosphate buffer solution.

FIG. 5A is a photomicrograph which depicts erythrocytes, where an osmotic pressure is changed in a 130% hypertonic solution.

FIG. 5B is a photomicrograph which depicts erythrocytes, where an osmotic pressure is changed in a 70% hypotonic solution.

FIG. 6A is a graph showing a permeation acceleration effect of incorporation of Arg Oligomer with Rhodamine to an erythrocyte.

FIG. 6B is a graph showing a permeation acceleration effect of incorporation of Arg Oligomer with Fluorescein to an erythrocyte.

FIG. 7 is a graph showing a permeation acceleration effect of incorporation of TAT peptide with Fluorescein to an erythrocyte.

FIG. 8 is a graph showing a membrane permeation acceleration effect of 1,3-butanediol.

FIG. 9 is a graph showing a membrane permeation acceleration effect of sodium thiocyanate.

FIG. 10 is a graph showing a membrane permeation acceleration effect of sucrose.

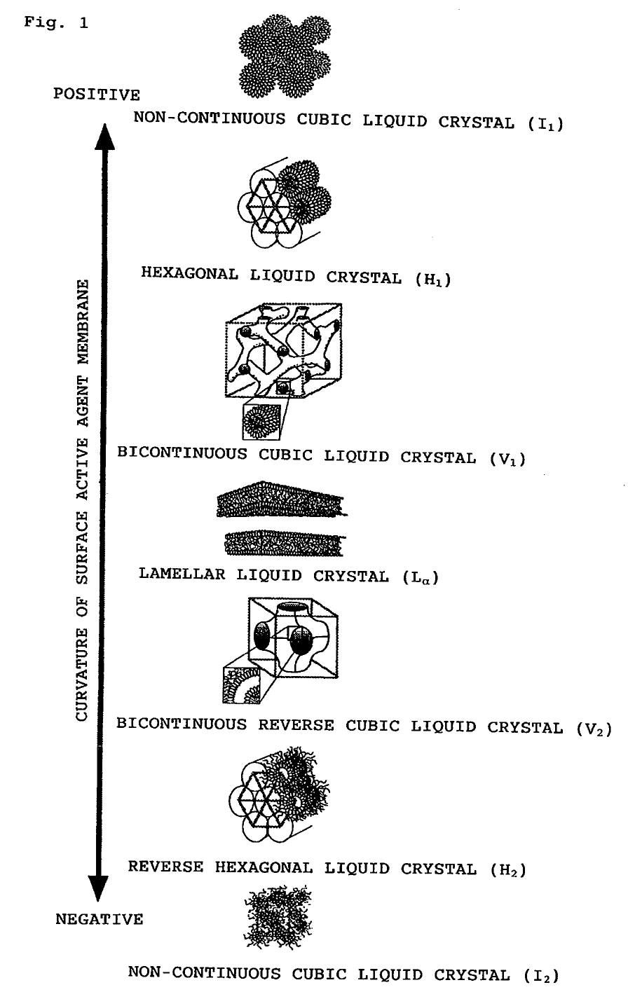

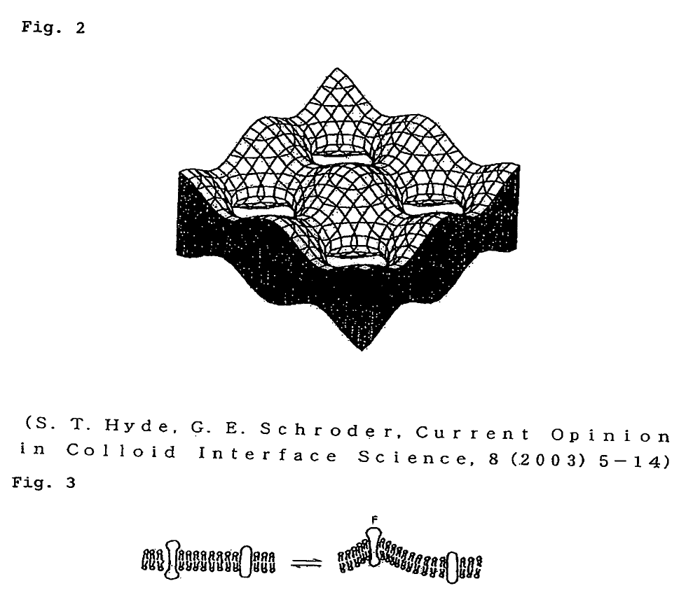

### DESCRIPTION OF THE PREFERRED EMBODIMENTS

The inventive membrane used is a lipid bimolecular membrane (hereinafter, sometimes simply referred to as a membrane). As the lipid bimolecular membrane, both a natural membrane and an artificial membrane may be used. Examples of natural membranes include a biological membrane, examples of which include a cell membrane, a nuclear membrane, a membrane enclosing a cell organelle, a retina, a lipid membrane in a stratum corneum, an enteric membrane, a blood-brain barrier, an intima of a blood vessel (such as a endothelial, connective tissue), a media of a blood vessel (such as smooth muscle, elastic fibers and collagen fibers), and an adventitia of a blood vessel (such as a loose connective tissue). Examples of cell membranes include membranes constituting a fibroblast, an epithelial cell, an endothelial cell, a hair matrix cell, a hair papilla cell, a nerve cell, a melanocyte, an epidermal keratinocyte, a Langerhans cell and a Merkel cell. Examples of artificial membranes include a liposome.

The membrane may contain other components, such as proteins and sugar chains, in addition to lipids if the membrane is a lipid bimolecular membrane.

The inventive membrane permeable substance is a substance which is able to penetrate the membrane; that preferably functions to change the curvature of the lipid bimolecular membrane in a direction making the membrane convex toward a side of said membrane donating the membrane permeable substance. Examples of the membrane permeable substance include a substance which is a peptide, a peptoid, or both, having from 2 to 30 side chains and/or groups selected from a guanidino side chain, an amidino side chain and an amino group, and contains an oligomer having at least one bond selected from an amide bond, a urethane bond, a polyester bond and a polyether bond. A target substance to penetrate through the membrane, such a drug, is attached to the peptide part, the peptoid part or the oligomer part of the membrane permeable substance, whereby the target substance can easily penetrate through the membrane to enter into the inside of the membrane.

The term “membrane permeable substance” referred in the invention means both the membrane permeable substance itself and the membrane permeable substance having a target substance to penetrate through the membrane, such as drug, attached thereto. Specific examples of the membrane permeable substance include Tat Peptide (48-60) (GRKKRRQRRRPPQC (SEQ ID NO. 1)), Penetratin (RQIKIWFQNRRMKWKK (SEQ ID NO. 2)), (Arg)₈ (SEQ ID NO. 3), (Lys)₈ (SEQ ID NO. 4) and amidated compounds thereof. A membrane permeability accelerating effect has been confirmed in various substances by utilizing the membrane permeable substance, and for example, Tat (37-72)-Anti-tetanus has been known. The amino acids constituting these peptides may be not only an L isomer but also a D isomer, which is expected to exert a similar effect as the L isomer. The use of a D isomer of the amino acid can improve the stability of the substance in the blood and the skin. Examples of a drug that is preferably attached to the membrane permeable substance include a peptidic drug, such as insulin, GLP-1(7-73), a somatotrophic hormone, a somatotrophic hormone releasing hormone, various kinds of antibodies, a cytokine and an enzyme, a polymer drug having low membrane permeability, such as cyclosporine, and a whitening agent having low skin permeability, but the invention is not limited to these.

The inventive method for controlling permeability of a membrane is a method for enhancing or suppressing permeability of a membrane to a membrane permeable substance by changing a membrane curvature at a site where the membrane and the membrane permeable substance are in contact with each other.

More specifically, examples of the method include:
a method of adding a third substance, which is capable of changing the physical properties of the membrane described later, to a composition containing the membrane permeable substance,
a method of adjusting an osmotic pressure of the composition, and
a method of incorporating a substance or a property into the membrane permeable substance for influencing the membrane curvature.

The site where the membrane and the membrane permeable substance are in contact with each other may be, in the case of percutaneous adsorption, on a lipid membrane in stratum corneum on the skin surface, on a membrane constituting a epidermal keratinocyte, on a dermal fibroblast, or on a melanocyte; in the case of transnasal adsorption by intranasal administration, on a membrane constituting nasal mucosa epithelial cell; and in the case of instillation, a corneal, as well as a scalp cell, a germinative cell, a hair papilla cell, or oral mucosa.

The change of the membrane curvature includes the change of the membrane curvature in a direction making the membrane convex or concave toward the side donating the membrane permeable substance. In the case where the membrane curvature is changed to be convex, the permeability of the membrane permeable substance is enhanced, and in the case where the curvature is changed to be concave, the permeability of the membrane permeable substance is suppressed.

In the inventive method, the change of the membrane curvature in a direction making the membrane convex toward the side donating the membrane permeable substance includes not only the case where the membrane is increased in curvature thereof to protrude into the side donating the membrane permeable substance, but also the case where a membrane having been made concave toward the side donating the membrane permeable substance is decreased in curvature thereof. Similarly, the change of the membrane curvature in a direction making the membrane concave toward the side donating the membrane permeable substance includes not only the case where the membrane curvature is decreased thereof to produce a depression from the side donating the membrane permeable substance, but also the case where a membrane curvature having been made convex toward the side donating the membrane permeable substance is decreased thereof.

Examples of the inventive method for changing the membrane curvature include a method of changing osmotic pressures of the solvents separated by the membrane. Specifically, in the case where the osmotic pressure on the side donating the membrane permeable substance is decreased compared to the osmotic pressure on the side accepting the membrane permeable substance, the membrane curvature is changed to make the membrane convex toward the side donating the membrane permeable substance to facilitate local phase change from the lamellar liquid crystal phase of the membrane to the cubic liquid crystal phase (V₁ phase). Therefore, the membrane permeability can be enhanced by decreasing the osmotic pressure on the side donating the membrane permeable substance. On the contrary, the membrane permeability can be inhibited by increasing the osmotic pressure on the side donating the membrane permeable substance.

Examples of the method for controlling the osmotic pressure include a method of disposing a composition containing the membrane permeable substance on the side donating the membrane permeable substance and then adding a substance increasing an osmotic pressure to the composition, and the method of diluting the composition to decrease the osmotic pressure. The substance increasing an osmotic pressure is not particularly limited and may be known water soluble substances in the broad sense of the term, and examples thereof include an inorganic electrolyte, such as sodium chloride and potassium chloride, an organic electrolyte, such as an amino acid, an organic acid and an organic amine, a saccharide, such as sucrose, a water soluble nonionic organic substance, such as urea, and a water soluble polymer. In this case, the composition containing the membrane permeable substance may further contain a pharmaceutically acceptable carrier, a vehicle and the like depending on the mode for using the composition.

Examples of the inventive method for changing the membrane curvature also include a method of changing at least one factor selected from the temperature, the electromagnetic field and the pH in the field where the membrane exists. The physical environments including the temperature and the magnetic field influence the motility of molecules constituting the membrane through the energy applied therefrom. As a result, the membrane permeability can be further effectively controlled solely thereby or by combination with other factors, such as the change of the osmotic pressure and the addition of the substance.

Examples of the inventive method for changing the membrane curvature further include a method of adding another substance, which is capable of changing the membrane curvature, to the composition containing the membrane permeable substance. Examples of the substance which is capable of changing the membrane curvature in this case include a substance that is in contact with single or plural water soluble sites of the amphiphilic substance constituting the membrane, and such substance locally increases the volume of the water soluble sites to make the curvature convex.

Specifically, examples of the substance include a substance having an ionic mutual action with the surface of the membrane, a substance having a hydrogen bond mutual action therewith, a substance having other physical mutual action therewith, and an amphiphilic substance having a large proportion of a hydrophilic part capable of changing the local membrane curvature to convex through mutual dissolution with the amphiphilic substance constituting the membrane. More specifically, it has been known that the following substances change the membrane curvature (as described in Langmuir, vol. 14, p. 5775-5781 (1998) and Langmuir, vol. 16, p. 8269 (2000)), i.e., a salt, such as sodium chloride (NaCl), sodium thiocyanate (NaSCN) and sodium sulfate (Na₂SO₄), and a polyol, such as glycerin, 1,3-butanediol and poly (ethylene glycol) 400 (PEG400), which may be used after appropriate selection depending on purposes. That is, the compound changing the curvature convex may be sodium thiocyanate (NaSCN), sodium sulfate (Na₂SO₄), 1,3-butanediol, sodium trifluoroacetate (CF₃COONa), N-acetyltyrosine, N-acetyltryptophan and N-acetylcysteine, and the compound changing the curvature concave may be sodium chloride (NaCl), poly (ethylene glycol) 400 (PEG400) and glycerin. Many of the substances that locally increase the volume of the water soluble sites to make the curvature convex simultaneously have the aforementioned function of changing the osmotic pressure as a water soluble substance.

Examples of the inventive method for changing the membrane curvature still further include a method of incorporating a substance or a property influencing the membrane curvature into the membrane permeable substance. The incorporation of a substance or a property influencing the membrane curvature into the membrane permeable substance may be attained, for example, by bonding a substance changing the curvature to a guanidyl group of arginine as a salt, and thereby the capability of the membrane permeable substance can be improved.

As having been described, the membrane permeability of the membrane permeable substance can be controlled by changing the membrane curvature.

The cell body enclosed with a membrane used in the inventive screening method means a cell body enclosed with a closed membrane separating the interior from the exterior, such as a liposome, a cell, a cell nucleus or an organelle, and preferred examples thereof include an erythrocyte, a ghost membrane thereof, and various kinds of liposomes. The size of the cell body enclosed with a membrane used in the invention is not particularly limited as far as the change of the membrane curvature can be measured, and is preferably about from 0.01 to 10 nm.

The solvent used in the screening method of the invention is not particularly limited so long as the cell bodies enclosed with a membrane can be dispersed therein, and in the case where a biological membrane permeable substance is to be screened, the solvent is preferably a hydrophilic solvent, and more preferably water, physiologic saline, a biological fluid and the like.

In the invention, the membrane curvature can be measured in the following manner. The cell bodies enclosed with a membrane are dispersed in a solvent and then observed with an optical microscope or a laser microscope to measure the radius of the cell bodies where they have a spherical shape, and a reciprocal number of the radius is obtained to determine the curvature. In the case where the cell bodies have an ellipsoidal shape, a shape having recesses on the center thereof like an erythrocyte, or an irregular shape like a fibrocyte, an image obtained with a microscope is applied to an ordinary image analysis system to process the shape parameters, whereby the curvature is determined. In the case where the cell bodies have a shape having recesses on the center thereof like an erythrocyte, the change in curvature can be determined by measuring the diameter thereof by using a Coulter Counter. Specifically, in the case where the membrane curvature of erythrocytes is changed by the osmotic pressure, the change in surface area of the erythrocytes by the osmotic pressure is small, and the average diameter of the erythrocytes is increased when the volume thereof is increased by the change in osmotic pressure. It is considered therefore that the increase in diameter by the change in osmotic pressure means the increase in volume thereof, and thus the increase in volume indicates that the membrane curvature of the erythrocytes is increased.

In one embodiment of the screening method of the invention, a test substance is added to a solvent dispersing cell bodies enclosed with a membrane, and the change in the membrane curvature of the cell bodies enclosed with a membrane before and after the addition is detected.

In the case where the membrane curvature after the addition of the test substance is larger than the membrane curvature before the addition, it is determined that the test substance has membrane permeability.

In another embodiment of the screening method of the invention, a solvent is retained in a first chamber and a second chamber separated from each other with a membrane, a test substance is added to the first chamber, and the change in the membrane curvature before and after the addition is detected. The chambers used in the invention are not particularly limited so long as the solvent can be separated with a membrane. Examples of the membrane used herein include the aforementioned various kinds of biological membranes, and an artificial membrane obtained by artificially orienting phospholipids to form a bimolecular membrane, such as a Langmuir-Blodgett (LB) membrane. A liposome is preferred owing to ease in preparation thereof. Upon observing the membrane curvature with a microscope or the like, it is determined that the test substance has membrane permeability in the case where the membrane curvature is changed to be convex toward the first chamber.

In still another embodiment of the invention, a test substance is added to a solvent dispersing cell bodies enclosed with a membrane, and the phase change of the membrane enclosing the cell bodies before and after the addition is detected. In a further embodiment of the invention, a solvent is retained in the first and the second chambers separated from each other with a membrane, a test substance is added to the first chamber, and the phase change of the membrane before and after the addition is detected.

In a still further embodiment of the invention, lipids constituting the membrane of the cell bodies are formed into a two-phase coexisting lamellar layer along with an aqueous phase, and the phase change is detected using the layer. Specific examples of the phase change of the membrane in these embodiments include a phase change from a lamellar liquid crystal phase to a cubic liquid crystal phase constituted by a lipid bimolecular membrane. In the case where a cubic liquid crystal phase is formed after the addition of the test substance, it is determined that the test substance has membrane permeability.

Examples of the method for directly detecting the phase change of the membrane include comparison between a tissue image observed under polarized or non-polarized conditions with a microscope for directly determining the attribution of the phase by observation of the phase thus formed with the naked eye and a stored image such as a photograph, observation with a transmission or scanning electron microscope, and determination of the phase type by addition of a pigment and observation of dissolution and diffusion thereof. Examples of the method indirectly detecting the phase change include a small angle X-ray scattering method showing the periodic structure characteristics in the self-organized structure of the amphiphilic substance constituting the membrane, a neutron small angle scattering method, a light-scattering method, an ESR (electron spin resonance) method as a spectroscopic measuring method showing the mobility and the flowability of the amphiphilic substance in the self-organized structure, an NMR (nuclear magnetic resonance) method, a determination by fluorescence spectroscopy, and a method utilizing a change in physicochemical property occurring upon the phase change from the lamellar phase to the cubic phase, which includes a measurement of the phase transition temperature by a DSC (differential scanning calorimetry) method, and a measurement of the thermal capacity.

In all the embodiments of the invention, the amount of the test substance added to the solvent can be appropriately determined depending on the properties of the test substance, and in general, the test substance is preferably added in an amount of about from 0.1 to 30% by weight.

### EXAMPLES

The invention will be described with reference to the following examples, but the examples are given only for explanatory use, and the invention is not construed as being limited thereto.

### Test Example 1

Influence of Osmotic Pressure on Curvature of Erythrocyte

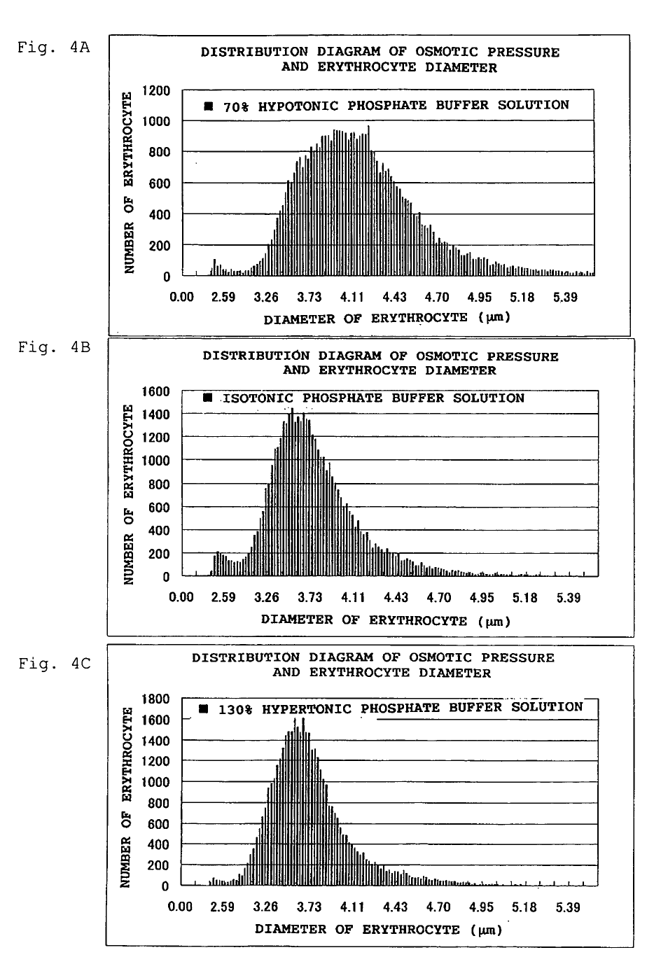

5 mL of preserved sheep blood (available from Japan Biomaterial Center Co., Ltd.) was placed in a tube, and after subjecting to centrifugal separation (4° C., 3,000 g, 3 min), the supernatant fluid was removed. 5 mL of physiological saline was added thereto, followed by lightly stirring, and then subjected to centrifugal separation (4° C., 3,000 g, 3 min). The same operation was repeated to obtain an erythrocyte pellet. 400 μL of an isotonic phosphate buffer solution was added to 400 μL of the erythrocyte pellet, and after stirring, the mixture was subjected to centrifugal separation (4° C., 3,000 g, 3 min), followed by removing the supernatant fluid. The same operation was repeated to obtain an erythrocyte pellet for evaluation. After replacing the solution in a cell counting analyzer (Coulter Counter® Model Z2 (produced by Beckman Coulter, Inc.)) by an isotonic phosphate buffer solution, the erythrocyte pellet for evaluation was diluted a hundred thousand times with an isotonic phosphate buffer solution to measure the diameter thereof, which was designated as the diameter of the erythrocytes in an isotonic solution. The solution in the Coulter Counter® is replaced by a hypotonic phosphate buffer solution (70% hypotonicity), and the diameter of the erythrocytes floating in the hypotonic phosphate buffer solution was measured in the same manner as in the case of the isotonic phosphate buffer solution. The diameter of the erythrocytes in a hypertonic phosphate buffer solution (130% hypertonicity) was also measured in the same manner. The distributions of the diameters of the erythrocytes in the isotonic, hypotonic and hypertonic phosphate buffer solutions are shown in FIGS. 4A, 4B, and 4C. As described above, the Coulter Counter® measures the volume of the erythrocyte and calculates the diameter from the volume under assumption that the erythrocyte has a spherical shape. Therefore, the increase in diameter means the increase in volume thereof. Erythrocytes in an isotonic solution are in a state of a negative curvature where the total membrane thereof is averagely recessed, and the surface area of the membrane of the erythrocyte is not largely changed. Therefore, the increase in volume means that the recession of the total membrane is expanded to render the membrane curvature directed in the positive direction. It is noted that the case where the surface area of the membrane is largely changed means breakage of the membrane itself, and the membrane of the erythrocyte is broken in a hypotonic solution of a 50% or more hypotonicity to cause complete hemolysis. Accordingly, it was understood that the use of a hypotonic osmotic pressure increased the diameter of the erythrocytes, and the membrane curvature of the erythrocytes was changed in the positive direction associated with the increase in volume of the erythrocytes.

### Test Example 2

Influence of Osmotic Pressure on Curvature of Erythrocyte

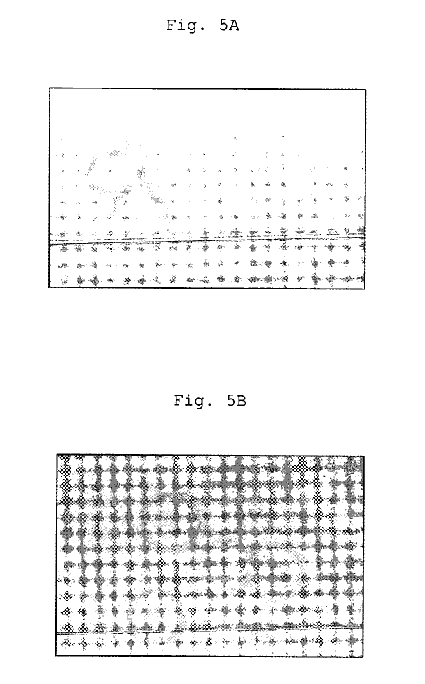

An erythrocyte pellet prepared in the same manner as in Test Example 1 was suspended in 70% hypotonic, isotonic and 130% hypertonic phosphate buffer solutions and observed for changes of the shape of the erythrocytes with a microscope (magnification: 3,500). The results obtained are shown in FIGS. 5A and 5B. It was understood that the use of a hypotonic osmotic pressure expanded the erythrocytes to render the curvature of the total membrane of the erythrocytes directed in the positive direction.

### Test Example 3

Influence of Osmotic Pressure on Membrane Permeation of Arg Oligomer into Erythrocyte

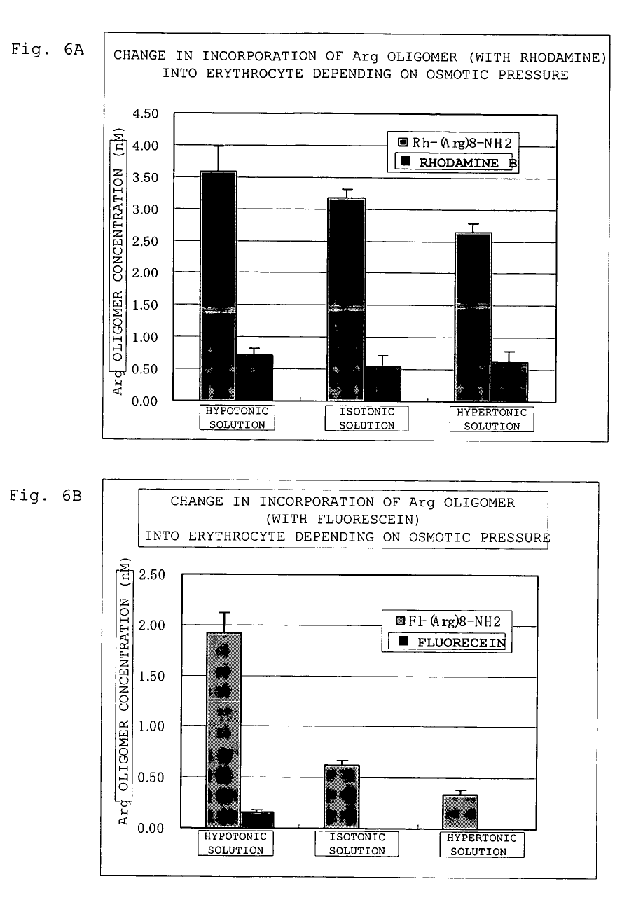

An erythrocyte pellet for evaluation was prepared in the same manner as in Test Example 1. An isotonic phosphate buffer solution containing 1 μM of fluorescent substance-added fluorescein-GABA-(Arg)₈-NH₂.9CF₃COOH or rhodamine-GABA-(Arg)₈-NH₂.9CF₃COOH (hereinafter, sometimes referred to as an Arg oligomer) as a membrane permeable substance was added to the erythrocyte pellet for evaluation, and after stirring, the solution was allowed to stand in an incubator at 37° C for 10 minutes. After allowing to stand for 10 minutes, the solution was subjected to centrifugal separation (4° C., 3,000 g, 3 min), and the supernatant fluid was removed, followed by rinsing with 400 μL of an isotonic phosphate buffer solution. The aforementioned operation was repeated twice. 200 μL of a surface active agent (1% Triton X-100) was added to the pellet, and after stirring, the mixture was subjected to centrifugal separation (4° C., 12,000 g, 5 min). The supernatant fluid was measured for fluorescence intensity with a microplate reader or a spectrophotofluorometer. The same experimentation was carried out except that the isotonic phosphate buffer was replaced with a 70% hypotonic phosphate buffer solution and a 130% hypertonic phosphate buffer solution to measure the incorporation amount of the Arg oligomer into the erythrocytes, respectively. The incorporation amounts of the Arg oligomer in the isotonic, hypotonic and hypertonic phosphate buffer solutions are shown in FIGS. 6A and 6B. For comparison, the same experimentation was carried out except that rhodamine or fluorescein was added instead of the membrane permeable substance. The results are also shown in FIGS. 6A and 6B. It was understood from FIGS. 6A and 6B that the amount of the Arg oligomer which penetrated into the erythrocytes was increased in comparison to the isotonic osmotic pressure by lowering the osmotic pressure to render the membrane curvature of the erythrocytes directed in the positive direction. It was understood, on the other hand, that in the case where rhodamine or fluorescein was added, the incorporation amounts were not substantially changed even through the osmotic pressure was changed.

### Test Example 4

Influence of Osmotic Pressure on Membrane Permeation of Tat Peptide (48-60) (GRKKRRQRRRPPQC (SEQ ID NO. 1)) into Erythrocyte

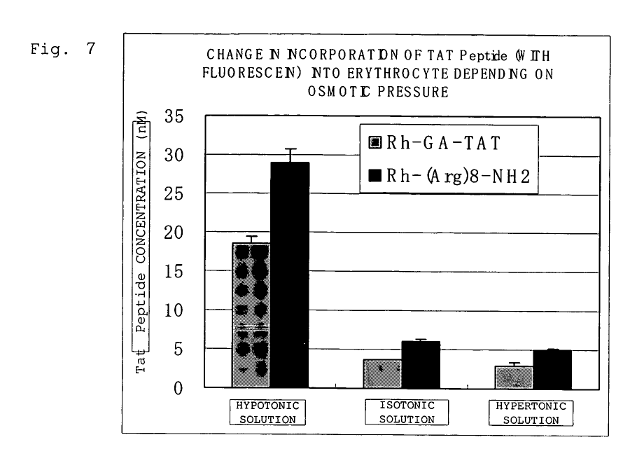

An erythrocyte pellet for evaluation was prepared in the same manner as in Test Example 1. An isotonic phosphate buffer solution containing 10 μM of fluorescent substance-added rhodamine-GABA-GRKKRRQRRRPPQC-NH₂.8CF₃COOH or rhodamine-GABA-(Arg)₈-NH₂.9CF₃COOH as a membrane permeable substance was added to the erythrocyte pellet for evaluation, and after stirring, the solution was allowed to stand in an incubator at 37° C for 10 minutes. After allowing to stand for 10minutes, the solution was subjected to centrifugal separation (4° C., 3,000 g, 3 min), and the supernatant fluid was removed, followed by rinsing with 400 μL of an isotonic phosphate buffer solution. The aforementioned operation was repeated twice. 200 μL of a surface active agent (1% Triton X-100) was added to the pellet, and after stirring, the mixture was subjected to centrifugal separation (4° C., 12,000 g, 5 min). The supernatant fluid was measured for fluorescence intensity with a microplate reader or a spectrophotofluorometer. The same experimentation was carried out except that the isotonic phosphate buffer was replaced with a 70% hypotonic phosphate buffer solution and a 130% hypertonic phosphate buffer solution to measure the incorporation amount of the Tat Peptide into the erythrocytes, respectively. The incorporation amounts of the Tat Peptide in the isotonic, hypotonic and hypertonic phosphate buffer solutions are shown in FIG. 7. It was understood from FIG. 7 that the amount of the Tat Peptide which penetrated into the erythrocytes was increased in comparison to the isotonic osmotic pressure by lowering the osmotic pressure to render the membrane curvature of the erythrocytes directed in the positive direction.

It was demonstrated by Test Examples 1 to 4 that the membrane permeability could be determined by detecting the membrane curvature. Therefore, novel cell membrane permeable substances can be screened from substances having an unknown function by detecting the curvature change of erythrocytes .

It was also understood from Test Examples 1 to 4 that the permeability of the membrane could be enhanced by changing the membrane curvature convex toward the side donating the membrane permeable substance.

### Test Example 5

Enhancing Effect of Incorporation of Arg Oligomer using Substance Positively Changing Curvature of Membrane

The enhancing effect of incorporation of an Arg oligomer was investigated upon adding 1,3-butanediol and sodium thiocyanate (NaSCN) as an enhancing substance, which were disclosed as substances positively changing the membrane curvature in Langmuir, vol. 14, p. 5775-5781 (1998) and Langmuir, vol. 16, p. 8269 (2000).

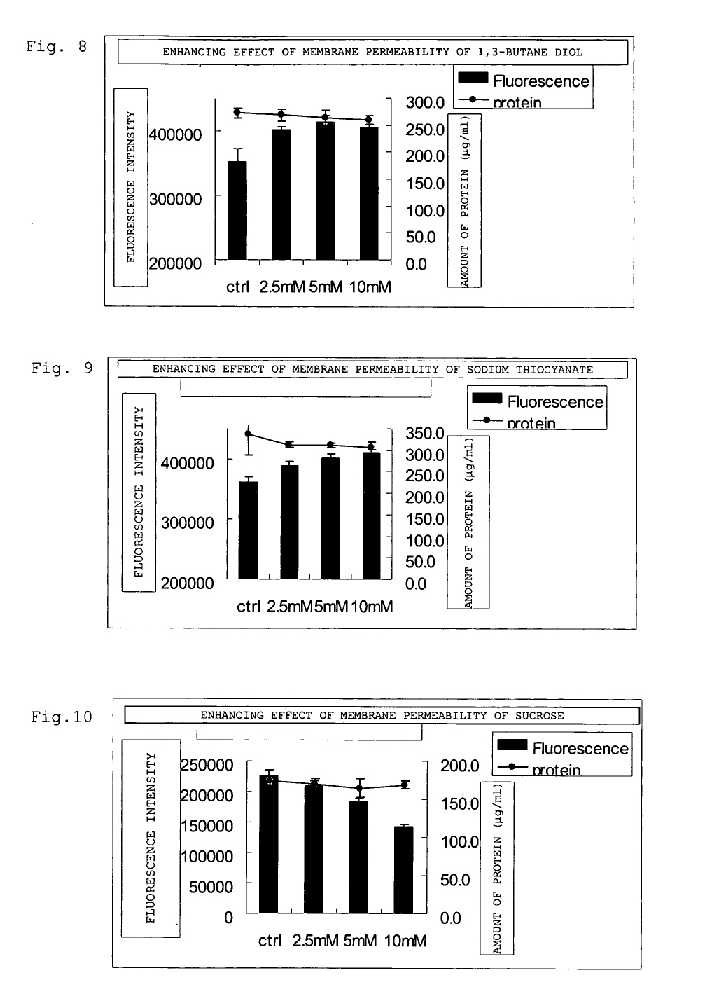

The enhancing effect of incorporation of an Arg oligomer was evaluated by using skin-related cells (dermal fibroblast). Dermal fibroblast (Fibrocell, produced by Kurabo Industries, Ltd.) was seeded on a 6-well plate in an amount of 3×10⁴ (cell/well) and cultivated with D-MEM Culture Medium (Dulbecco's modified Eagle medium, containing 10% serum, penicillin: 50 U/mL, streptomycin: 50 μg/mL) for 2 days. After cultivating for 2 days, the culture medium was removed from the wells of the 6-well plate, and D-MEM Culture Medium adjusted to contain 1,3-butanediol in a concentration of 2.5, 5 or 10 mM was added. After incubating for 5 minutes (37° C., 5% CO₂), an Arg oligomer was added to the respective plates to a concentration of 1 μM and incubated for 10 minutes. After 10 minutes, the culture medium was removed by suction, and the wells were rinsed twice with an isotonic phosphate buffer solution. 500 μL of 1% Triton X-100 was added to the respective wells, followed by shaking at room temperature under light shielding for 1 hour, to lyse the cells. After thoroughly stirring by pipetting, the contents of the respective cells were transferred by 200 μL to a 96-well black cell, and fluorescence of 535 nm was measured under excitation with a spectrophotofluorometer (microplate reader, Wallac 1420 ARVOsx) at 485 nm. The addition of 2.5 mM of 1,3-butanediol increased the fluorescence intensity by about 15% in comparison to the case where no 1,3-butanediol was added. The addition of 2.5 mM of sodium thiocyanate increased the fluorescence intensity by about 10%, and the addition of 10 mM thereof increased the fluorescence intensity by about 15%. According to the aforementioned literatures, 1,3-butanediol and sodium thiocyanate are compounds changing the membrane curvature to convex, and it is considered that the change of the membrane improves the membrane permeability of the Arg oligomer. The results of the experimentation are shown in FIGS. 8 and 9.

### Test Example 6

Suppressing Effect of Incorporation of Arg Oligomer using Substance Negatively Changing Membrane Curvature

The suppressing effect of incorporation of an Arg oligomer was investigated upon adding sucrose as a permeation suppressing substance, which was disclosed as a substance negatively changing the membrane curvature in Biochimica et Biophysica Acta (BBA)—Biomembranes, vol. 1285, Issue 1, p. 109-122 (1996). The inventors confirmed that a cubic phase of lecithin/sodium cholate system was constituted by utilizing lecithin (phosphatidylcholine) as a major constitutional component of the phospholipid constituting a cell membrane, and the cubic phase was changed to a hexagonal phase by adding sucrose thereto (as described in Biochim. Biophys. Acta., vol. 125, p. 563-580 (1966)). The phase change was determined by a change of the shape observed by a polarization microscope, the presence or absence of polarization with a polarizing film (a lamellar phase and a hexagonal phase were viewed brightly owing to the polarization thereof, but a cubic phase is viewed darkly), or a difference in spectrum patterns obtained by small angle X-ray scattering. The cubic phase was formed by mixing 22.5% by weight of water, 50.4% by weight of phosphatidylcholine and 27.1% by weight of sodium cholate at 25° C. When sucrose was added to the cubic phase in a proportion of 3.5% by weight or more, the cubic phase was changed to a lamellar phase. It has been known that the direction of this phase change is induced by a negative change of the membrane curvature. Accordingly, it was confirmed that sucrose was a substance that negatively changed the membrane curvature. The suppressing effect of incorporation of an Arg oligomer was investigated upon adding sucrose, which negatively changed the membrane curvature in the lecithin/sodium cholate system.

The suppressing effect of incorporation of an Arg oligomer was evaluated by using skin-related cells (dermal fibroblast). Dermal fibroblast (Fibrocell, produced by Kurabo Industries, Ltd.) was seeded on a 6-well plate in an amount of 3×10⁴ (cell/well) and cultivated with D-MEM Culture Medium (Dulbecco's modified Eagle medium, containing 10% serum, penicillin: 50 U/mL, streptomycin: 50 μg/mL) for 2 days. After cultivating for 2 days, the culture medium was removed from the wells of the 6-well plate, and D-MEM Culture Medium adjusted to contain sucrose in a concentration of 2.5, 5 or 10 mM was added. After incubating for 5 minutes (37° C., 5% CO₂), an Arg oligomer was added to the respective plates to a concentration of 1 μM and incubated for 10 minutes. After lapsing 10 minutes, the culture medium was removed by suction, and the contents of the wells were rinsed twice with an isotonic phosphate buffer solution. 500 μL of 1% Triton X-100 was added to the respective wells, followed by shaking at room temperature under light shielding for 1 hour, to lyse the cells. After thoroughly stirring by pipetting, the contents of the respective wells were transferred by 200 μL to a 96-well black cell, and fluorescence of 535 nm was measured under excitation with a spectrophotofluorometer (microplate reader, Wallac 1420 ARVOsx) at 485 nm. The addition of sucrose decreased the fluorescence intensity depending upon the concentration of sucrose by 8% with a sucrose concentration of 2.5 mM, 19% with 5 mM and 37% with 10 mM, in comparison to the case where no sucrose was added. According to the aforementioned experimentation, sucrose is a compound changing the membrane curvature to concave, and it is considered that the change of the membrane suppresses the membrane permeability of the Arg oligomer. The results of the experimentation are shown in FIG. 10.

### Application Example 1

An erythrocyte pellet for evaluation is prepared in the same manner as in Test Example 1 and floated in 70% hypotonic, isotonic and 130% hypertonic phosphate buffer solutions. An isotonic phosphate buffer solution, a 70% hypotonic phosphate buffer solution and a 130% hypertonic phosphate buffer solution each containing from 0.01 to 100 μM of a subject compound are prepared. The subject compound is appropriately selected from the compound libraries and the like, and it is preferred to prepare plural solutions with different concentrations. The subject compound may be in a detectable form modified with an appropriate chemical substance (such as a fluorescent substance and a radio isotope), or may be directly measured with NMR, LC-MS/MS or the like.

The hypotonic, isotonic and hypertonic subject compound solutions each is added to the hypotonic, isotonic and hypertonic erythrocyte pellets for evaluation, respectively, and the incorporated amount of the subject compound into the erythrocytes in the hypotonic, isotonic or hypertonic solution is determined by measuring the amount of the compound incorporated in the erythrocytes, or by calculating the incorporated amount of the compound from the amount of the compound that is not incorporated therein. In the case where the subject compound is increased in incorporated amount in the hypotonic solution and decreased in incorporated amount in the hypertonic solution, as similar to the incorporation behavior of a Arg oligomer in Test Example 3, the compound is determined as being positively changing the curvature of the erythrocyte membrane, and thus it can be designated as a candidate compound of a novel cell membrane permeable substance.

### Application Example 2

Other application examples of the invention include the following.

1. A sublingual tablet containing insulin bound with an Argoligomer (drug) as a permeable medical substance is dissolved in a prescribed amount of water upon dose to form a hypotonic permeable drug solution, and the necessary concentration of insulin can be effectively absorbed through sublingual mucosa for controlling the postcibal blood glucose level of a diabetic patient. The drug is not necessarily stabilized in the solution and is convenient for carrying.

2. A medical agent containing insulin bound with an Arg oligomer (drug) as a permeable medical substance and an acylglutamate salt as a permeation accelerating substance is directly coated on a skin of a diabetic patient before or after meal for controlling the postcibal blood glucose level, or in alternative, a medium is impregnated with the medical agent and attached to the skin of the diabetic patient, whereby the necessary concentration of insulin can be effectively absorbed percutaneously.

1. A method for controlling membrane permeability of a membrane permeable substance, comprising the step of changing the curvature of the membrane at a site where the membrane and the membrane permeable substance are in contact with each other.

2. A method for enhancing permeability of a membrane, comprising the step of changing the curvature of the membrane at a site where the membrane and a membrane permeable substance are in contact with each other, in a direction making the membrane convex toward a side of said membrane donating a membrane permeable substance.

3. A method for suppressing permeability of a membrane, comprising the step of changing the curvature of the membrane at a site where the membrane and a membrane permeable substance are in contact with each other, in a direction making the membrane concave toward a side donating a membrane permeable substance.

4. The method as claimed in claim 1, wherein the method comprises the step of changing the curvature of the membrane by changing at least one factor selected from the group consisting of an osmotic pressure, a temperature, an electromagnetic field and pH.

5. The method as claimed in claim 1, wherein the method comprises the step of adding a substance changing the curvature of the membrane to a composition containing the membrane permeable substance.

6. The method as claimed in claim 2, wherein the method comprises the step of adding a substance changing the curvature of the membrane to a composition containing the membrane permeable substance.

7. The method as claimed in claim 3, wherein the method comprises the step of adding a substance changing the curvature of the membrane to a composition containing the membrane permeable substance.

8. The method as claimed in claim 5, wherein the substance changing the curvature of the membrane is at least one selected from the group consisting of a water soluble substance, an oil soluble substance and an amphiphilic substance.

9. The method as claimed in claim 6, wherein the substance changing the curvature of the membrane is a substance which changes the curvature of the membrane to convex, and wherein said substance is at least one selected from the group consisting of sodium thiocyanate, sodium sulfate and 1,3-butanediol.

10. The method as claimed in claim 7, wherein the substance changing the curvature of the membrane is a substance which changes the curvature of the membrane to concave, and wherein said substance is at least one selected from the group consisting of sodium chloride, poly (ethylene glycol) 400 and glycerin.

11. The method as claimed in claim 1, wherein the membrane is a biological membrane and wherein the membrane is at least one selected from the group consisting of a cell membrane, a nuclear membrane, a membrane enclosing a cell organelle, a retina, a lipid membrane in a stratum corneum, an enteric membrane, a blood-brain barrier, an intima of a blood vessel, a media of a blood vessel and an adventitia of a blood vessel.

12. The method as claimed in claim 11, wherein the cell membrane is a membrane constituting at least one cell selected from the group consisting of a fibroblast, an epithelial cell, an endothelial cell, a hair matrix cell, a hair papilla cell, a nerve cell, a melanocyte, a epidermal keratinocyte, a Langerhans cell and a Merkel cell.

13. The method as claimed in claim 1, wherein the membrane permeable substance is a peptide, a peptoid, or both, having from 2 to 30 side chains and/or groups selected from a guanidino side chain, an amidino side chain and an amino group, and
wherein the membrane permeable substance is a substance containing an oligomer having at least one bond selected from the group consisting of an amide bond, a urethane bond, a polyester bond and a polyether bond.

14. The method as claimed in claim 1, wherein the membrane permeable substance is a substance having attached thereto a peptide selected from the group consisting of insulin, GLP-1 (3-37), a somatotrophic hormone, a somatotrophic hormone releasing hormone, an antibody, cytokine and an enzyme.

15. A method for screening a membrane permeable substance, comprising the steps of:
adding a test substance to a solvent dispersing cell bodies enclosed with a membrane; and
detecting the curvature change and/or a phase change of the membrane of the cell bodies enclosed by a membrane before and after the addition of the test substance.

16. The method as claimed in claim 15, wherein in a case where the curvature of the membrane after the addition of the test substance is larger than the curvature of the membrane before the addition, or in a case where the formation of a cubic liquid crystal phase is detected as a phase change after the addition of the test substance, it is determined that the test substance has membrane permeability.

17. A method for screening a membrane permeable substance, comprising the steps of:
retaining a solvent in first and second chambers separated from each other with a membrane;
adding a test substance to the first chamber; and
detecting the curvature change and/or a phase change of the membrane before and after the addition of the test substance.

18. The method as claimed in claim 17, wherein in a case where the curvature of the membrane is changed to be convex toward the first chamber, or in a case where the formation of a cubic liquid crystal phase is detected as a phase change after the addition of the test substance, it is determined that the test substance has membrane permeability.

19. The method as claimed in claim 15, wherein the change of the curvature of the membrane is detected by using a microscope.

20. The method as claimed in claim 15, wherein the change of the curvature of the membrane is detected by using a Coulter Counter.

21. The method as claimed in claim 15, wherein the change of the phase of the membrane is detected by X-ray diffraction or a change in phase transition temperature of the membrane.

22. The method as claimed in claim 17, wherein the change of the phase of the membrane is detected by X-ray diffraction or a change in phase transition temperature of the membrane.

23. The method as claimed in claim 15, wherein the cell body enclosed with a membrane is selected from the group consisting of a liposome, an erythrocyte, a leukocyte, a lymphocyte, a cell such as an epithelial cell or a fibrocyte fibroblast, a cell nucleus and an organelle.

24. The method as claimed in claim 15, wherein the membrane is at least one selected from the group consisting of a biological membrane constituting a lipid bimolecular membrane including a cell membrane, a nuclear membrane, a membrane enclosing a cell organelle, a retina, a lipid membrane in a stratum corneum, an enteric membrane, a blood-brain barrier, an intima of a blood vessel, a media of a blood vessel, and an adventitia of a blood vessel, and an artificial membrane.

25. The method as claimed in claim 17, wherein the membrane is at least one selected from the group consisting of a biological membrane constituting a lipid bimolecular membrane including a cell membrane, a nuclear membrane, a membrane enclosing a cell organelle, a retina, a lipid membrane in a stratum corneum, an enteric membrane, a blood-brain barrier, an intima of a blood vessel, a media of a blood vessel, and an adventitia of a blood vessel, and an artificial membrane.

26. The method as claims in claim 24, wherein the cell membrane is a membrane constituting at least one cell selected from the group consisting of a fibroblast, an epithelial cell, an endothelial cell, a hair matrix cell, a hair papilla cell, a nerve cell, a melanocyte cell, a epidermal keratinocyte, a Langerhans cell and a Merkel cell.

27. The method as claims in claim 25, wherein the cell membrane is a membrane constituting at least one cell selected from the group consisting of a fibroblast, an epithelial cell, an endothelial cell, a hair matrix cell, a hair papilla cell, a nerve cell, a melanocyte cell, a epidermal keratinocyte, a Langerhans cell and a Merkel cell.
